SEQUENCE LISTING (12 sequences)

SEQ ID NO: 1 - DNA, 2520 bp, Homo sapiens
atgggatcgctgcttgcgctcctggcactgctgccgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggatcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgaggcctctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgaacaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatggcagtgggcgctatcgctaccagaaggtgggctactgggcagaaggcttgactctggacaccagcctcatcccatgggcctcaccgtcagccggccccctggccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctgtttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatctcgggccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacaatgtgctcctcatcgcgctctgcacgctttatgccttcaatactcgcaagtgccccgaaaacttcaacgaggccaagttcattggcttcaccatgtacaccacctgcatcatctggctggcattgttgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctt

SEQ ID NO: 2 - protein, 840 aa, Homo sapiens
MGSLLALLALLPLWGAVAEGPAKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTEASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDGSGRYRYQKVGYWAEGLTLDTSLIPWASPSAGPLAASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALISGQLLIVVAWLVVEAPGTGKETAPERREVVTLRCNHRDASMLGSLAYNVLLIALCTLYAFNTRKCPENFNEAKFIGFTMYTTCIIWLALLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSL

SEQ ID NO: 3 - DNA, 20 bp, Homo sapiens
aagtttgatggcagtgggcg

SEQ ID NO: 4 - protein, 10 aa, Homo sapiens
NVKFDGSGRY

SEQ ID NO: 5 - DNA, 25 bp, Homo sapiens
agaggactgtggtcctgtcaatgag

SEQ ID NO: 6 - DNA, 25 bp, Homo sapiens
acacataggtccagttgaagaagcg

SEQ ID NO: 7 - DNA, 25 bp, Homo sapiens
tccaagatcatgtttgtggtcaatg

SEQ ID NO: 8 - DNA, 25 bp, Homo sapiens
agtgaattcgtccaatcggtactca

SEQ ID NO: 9 - DNA, 25 bp, Homo sapiens
ctgcacgctttatgccttcaatact

SEQ ID NO: 10 - DNA, 24 bp, Homo sapiens
cattgcaaacagtggggacaaact

SEQ ID NO: 11 - DNA, 25 bp, Homo sapiens
atgggatcgctgcttgcgctcctgg

SEQ ID NO: 12 - DNA, 27 bp, Homo sapiens
aagcgatgacgttgtcgagtcctcacg

(19) United States
(12) Patent Application Publication
(10) Pub. No.: US 2004/0185501 A1
(43) Pub. Date: Sep. 23, 2004

(54) Alternatively spliced isoform of human GRM2

(76) Inventors: Jason M. Johnson, Seattle, WA (US); Philip W. Garrett-Engele, Seattle, WA (US)

Correspondence Address: R. Douglas Bradley, Merck & Co., Inc. - Patent Dept., P.O. Box 2000, RY60-30, Rahway, NJ, 07065-0907, US

(21) Appl. No.: 10/657,552

(22) Filed: Sep. 8, 2003

Related U.S. Application Data
(60) Provisional application No. 60/409,094, filed on Sep. 9, 2002.

(51) Int. Cl. G01N 33/53; C07H 21/04; C07K 14/705

(52) U.S. Cl. 435/7.1; 435/69.1; 435/320.1; 435/325; 530/350; 536/23.5

(57) ABSTRACT

The present invention features nucleic acids and polypeptides encoding a novel splice variant isoform of a human metabotropic glutamate receptor 2 gene (GRM2). The polynucleotide sequence of GRM2sv1 is provided by SEQ ID NO 1. The amino acid sequence for GRM2sv1 is provided by SEQ ID NO 2. The present invention also provides methods for using GRM2sv1 polynucleotides and proteins to screen for compounds that bind to or interact with GRM2sv1.

This application claims priority to U.S. Provisional Patent Application Serial No. 06/409,094 filed on Sep. 9, 2002, which is incorporated by reference herein in its entirety.

### BACKGROUND OF THE INVENTION

The references cited herein are not admitted to be prior art to the claimed invention.

Glutamate is the major excitatory neurotransmitter in mammalian central nervous systems (CNS). Interaction of glutamate with its receptors plays an important role in many neuronal processes, including long-term potentiation, fast excitatory synaptic transmission, learning and memory. In addition, glutamate serves as a neurotoxic agent that plays a critical role in neurological disorders (Hermans and Challis, 2001 Biochem. J., 359:465-484).

Glutamate exerts its neurotransmitter effects through two types of receptors: ionotropic and metabotropic. The ionotropic glutamate receptors contain cation-specific, ligand-gated ion channels. In contrast, the metabotropic glutamate receptors are G-protein coupled receptors that exert their effects through secondary effectors such as adenylyl cyclase, phospholipase C and plasma membrane ion channels selective for Ca²⁺ and K⁺ (Dingledine et al., 1999 Pharmacol. Rev. 51:7-61).

There are eight distinct subtypes of metabotropic glutamate receptors (mGluR1-mGluR8). These subtypes have been classified into three groups (I, II, and III) based on their sequence similarities, pharmacological properties, and preferred signal transduction mechanisms (Pin and Duvoisin, 1995 Neuropharmacology, 34:1-26). Each receptor has a large extracellular glutamate binding domain, a cysteine-rich region, a seven transmembrane domain, and an intracellular domain that couples and activates G-proteins (Hermans and Challiss, 2001 Biochem. J., 358: 465-484).

A group II human metabotropic glutamate receptor was isolated from human hippocampus and fetal brain cDNA libraries using the rat mGluR2 cDNA (Flor et al., 1995 Eur. J. Neurosci. 7:622-629). This human metabotropic glutamate receptor 2 gene, designated GRM2, contains five coding exons and maps to chromosome 3 (Marti et al., 2002 Am. J. Med. Gen., 114:12-12; Joo et al., 2001 Molecular Psychiatry, 6: 186-192). Once bound by glutamate, GRM2 produces its effects by negatively coupling to adenylate cyclase to inhibit cyclic AMP (cAMP) formation. This results in a decrease in the active form of transcription factors containing cAMP response elements, leading to changes in gene expression. GRM2 protein localizes to presynaptic neurons, thereby implicating the protein in the inhibition of synaptic transmissions (Sharpe et al., 2002 British Journal of Pharmacology, 135:1255-1262).

The effects of agonists on GRM2 and other group II metabotropic glutamate receptors indicate that these receptors play major roles in many important clinical diseases (for a review see Conn and Pin, 1997 Annu. Rev. Pharmacol. Toxicol. 37:205-237). First, group H metabotropic receptor agonists possess anti-epilepsy activity (Moldrich et al., 2001 Neuropharmacology 41:8-18), indicating that GRM2 may be an important target for the development of anticonvulsant drugs. Second, agonists of group II metabotropic receptors protect cultured neurons against β-amyloid peptide-induced apoptosis (Copani et al., 1995 Mol. Pharmacol., 47: 890-897), suggesting that GRM2 agonists have the potential to reduce the progression of Alzheimer's disease. Third, a group II agonist, has exhibited antipsychotic action in a rat schizophrenia model (Cartmell et al., 2000 Eur. J. Pharmacol., 400:221-224; Cartmell et al., 1999 The Journal of Pharmacology and Experimental Therapeutics, 291:161-170; Moghaddam and Adams, 1998 Science, 281:1349-1352). Finally, group II agonists also reduce the hyperexcitable states underlying the diseases hyperalgesia and allodynia, which result in undue sensitivity to pain (Sharpe et al., 2002 British Journal of Pharmacology, 135:2355-1262). This result suggests that drugs targeted to GRM2 may be useful in the treatment of acute and chronic pain. Therefore, GRM2 may serve as an important target for the development of many beneficial drugs.

Because of the importance of GRM2 as a drug target and its myriad of roles in neurological function and disease, there is a need in the art for compounds that selectively bind to or interact with isoforms of human GRM2. The present invention is directed towards a novel GRM2 isoform (GRM2sv1) and uses thereof.

### SUMMARY OF THE INVENTION

RT-PCR has been used to identify and confirm the presence of a human splice variant of GRM2 mRNA. More specifically, the present invention features polynucleotides encoding GRM2sv1 and GRM2sv1 polypeptides. The cDNA sequence encoding GRM2sv1 is provided by SEQ ID NO 1. The amino acid sequence for GRM2sv1 is provided by SEQ ID NO 2.

Thus, a first aspect of the present invention describes a purified GRM2sv1 encoding nucleic acid. The nucleic acid comprises SEQ ID NO 1 or the complement thereof. Reference to the presence of one region does not indicate that another region is not present. For example, in different embodiments the inventive nucleic acid can comprise, consist or consist essentially of a nucleic acid encoding for SEQ ID NO 2 and can comprise, consist or consist essentially of the nucleic acid sequence of SEQ ID NO 1.

Another aspect of the present invention describes a purified GRM2sv1 polypeptide. The polypeptide can comprise, consist, or consist essentially of the amino acid sequence of SEQ ID NO 2.

Another aspect of the present invention describes an expression vector. The expression vector comprises a nucleotide sequence encoding a polypeptide comprising or consisting of SEQ ID NO 2, wherein the nucleotide sequence is transcriptionally coupled to an exogenous promoter. Alternatively, the nucleotide sequence comprises, consists, or consists essentially of SEQ ID NO 1 and is transcriptionally coupled to an exogenous promoter.

Another aspect of the present invention describes a recombinant cell comprising an expression vector comprising or consisting of the above-described sequences and the promoter is recognized by an RNA polymerase present in the cell. Another aspect of the present invention, describes a recombinant cell made by a process comprising the step of introducing into the cell an expression vector comprising a nucleotide sequence comprising or consisting of SEQ ID NO 1, or a nucleotide sequence encoding a polypeptide comprising, consisting, or consisting essentially of an amino acid sequence of SEQ ID NO 2, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing a GRM2sv1 polypeptide comprising SEQ ID NO 2. The method involves the step of growing a recombinant cell containing an inventive expression vector under conditions wherein the polypeptide is expressed from the expression vector.

Another aspect of the present invention features a purified antibody preparation comprising an antibody that binds selectively to GRM2sv1 as compared to one or more GRM2 isoform polypeptides that is not GRM2sv1.

Another aspect of the present invention provides a method of screening for a compound that binds to GRM2sv1 or a fragment thereof. The method comprises the steps of: (a) expressing a polypeptide comprising the amino acid sequence of SEQ ID NO 2 or fragment thereof from recombinant nucleic acid; providing to said polypeptide a labeled GRM2 ligand that binds to said polypeptide and a test preparation comprising one or more test compounds; and measuring the effect of said test preparation on binding of said labeled GRM2 ligand to said polypeptide.

In another embodiment of the method, a compound is identified that binds selectively to GRM2sv1 as compared to one or more GRM2 isoform polypeptides that are not GRM2sv1. This method comprises the steps of: providing a GRM2sv1 polypeptide comprising SEQ ID NO 2; providing a GRM2 isoform polypeptide that is not GRM2sv1, contacting said GRM2sv1 polypeptide and said GRM2 isoform polypeptide that is not GRM2sv1 with a test preparation comprising one or more test compounds; and determining the binding of said test preparation to said GRM2sv1 polypeptide and said GRM2 isoform polypeptide that is not GRM2sv1, wherein a compound that binds said GRM2sv1 polypeptide but does not bind said GRM2 isoform polypeptide that is not GRM2sv1 is a compound that selectively binds said GRM2sv1 polypeptide.

In another embodiment of the method, a compound is identified that binds selectively to one or more GRM2 isoform polypeptides that are not GRM2sv1 as compared to GRM2sv1. This method comprises the steps of: providing a GRM2sv1 polypeptide comprising SEQ ID NO 2; providing a GRM2 isoform polypeptide that is not GRM2sv1, contacting said GRM2sv1 polypeptide and said GRM2 isoform polypeptide that is not GRM2sv1 with a test preparation comprising one or more test compounds; and determining the binding of said test preparation to said GRM2sv1 polypeptide and said GRM2 isoform polypeptide that is not GRM2sv1, wherein a compound that binds said GRM2 isoform polypeptide that is not GRM2sv1 but does not bind said GRM2sv1 polypeptide is a compound that selectively binds to one or more GRM2 isoform polypeptides that are not GRM2sv1.

In another embodiment of the invention, a method is provided for screening GRM2sv1 activity comprising the steps of: contacting a cell expressing a recombinant nucleic acid encoding GRM2sv1 wherein said GRM2sv1 has an amino acid sequence comprising SEQ ID NO 2 with a test preparation comprising one or more test compounds; and measuring the effect of said test preparation glutamate binding activity, or cAMP levels as a result of activity of said GRM2sv1.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE FIGURES

FIG. 1 presents the results of RT-PCR assays using polyA mRNA obtained from ten human tissue samples. A list of the ten human polyA mRNA samples in lanes 1-10 is presented in Table 1.

FIG. 2A illustrates the exon structure of GRM2 mRNA corresponding to the known long reference form of GRM2 mRNA (labeled NM_{—}000839). FIG. 2B illustrates the inventive short form splice variant of GRM2 mRNA (labeled GRM2sv1). The small arrows above exons 2 and 3 show the positions of the oligonucleotide primers used to perform RT-PCR assays to confirm the exon structure of GRM2 mRNA in two human tissue samples (see FIG. 1). The nucleotide sequences shown in boxes below the exon structure diagrams of the GRM2 and GRM2sv1 mRNAs depict the nucleotides sequences of the exon junctions resulting from the splicing of exon 2 to exon 3 in the case of the GRM2 mRNA (FIG. 2A); and the splicing of exon 2 to nucleotide 97 of exon 3 in the case of GRM2sv1 mRNA (FIG. 2B). In FIG. 2A, the nucleotides shown in italics represent 20 nucleotides at the 3′ end of exon 2 and the nucleotides shown in bold represent 20 nucleotides at the 5′ end of exon 3. In FIG. 2B, the nucleotides shown in italics represent 20 nucleotides at the 3′ end of exon 2 and the nucleotides shown in bold represent 20 nucleotides at the 5′ end of the truncated form of exon 3 that is missing the first 96 nucleotides of exon 3.

Definitions

Unless defined otherwise, all technical and scientific terms used herein have the meaning commonly understood by one of ordinary skill in the art to which this invention belongs.

As used herein, “GRM2” refers to a human glutamate receptor 2 protein (NP_{—}000830). In contrast, the term “GRM2 isoform” is meant to include GRM2 and GRM2 isoform proteins having amino acid sequences that are not identical to NP_{—}000830, e.g., GRM2 polymorphisms, splice variants and the like.

As used herein, “GRM2sv1” refers to a splice variant isoform of human GRM2 protein having an amino acid sequence set forth in SEQ ID NO 2.

As used herein, “GRM2” refers to polynucleotides encoding GRM2 (NM_{—}000839). In contrast, the term “GRM2 isoform” is meant to include polynucleotides encoding GRM2 and GRM2 isoform proteins and having nucleotide sequences that are not identical to NM_{—}000839, e.g., single nucleotide polymorphisms, splice variants and the like.

As used herein, “GRM2sv1” refers to polynucleotides encoding GRM2sv1 having an amino acid sequence set forth in SEQ ID NO 2.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻⁹ M.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

The present invention relates to the amino acid sequence of human GRM2sv1, which is a splice variant isoform of GRM2, and to nucleotide sequences encoding this protein. SEQ ID NO 1 is a polynucleotide sequence containing a full open reading frame that encodes GRM2sv1 protein (SEQ ID NO 2).

GRM2sv1 polynucleotides and GRM2sv1 proteins, as exemplified and enabled herein include a number of specific, substantial and credible utilities. For example, GRM2sv1 encoding nucleic acids were identified in a mRNA sample obtained from a human source (see Example 1-3). Such nucleic acids can be used as hybridization probes to distinguish between cells that produce GRM2sv1 transcripts from human or non-human cells (including bacteria) that do not produce such transcripts. Similarly, antibodies specific for GRM2sv1 can be used to distinguish between cells that express GRM2sv1 from human or non-human cells (including bacteria) that do not express GRM2sv1.

GRM2 is an important drug target for compounds that have therapeutic value in the management of many neurological disorders. Given the importance of GRM2 activity to the therapeutic management of these diseases, it is of value to identify GRM2 isoforms and identify GRM2-ligand compounds that are isoform-specific as well as other compounds that are effective ligands for many GRM2 isoforms. In particular, it may be important to identify compounds that are effective inhibitors or activators of a specific GRM2 isoform activity, yet do not bind to a plurality of other GRM2 isoforms. Compounds that bind to multiple GRM2 isoforms may require higher drug doses to saturate multiple GRM2 isoform binding sites, and thereby result in a greater likelihood of secondary non-therapeutic side effects. For the foregoing reasons, GRM2sv1 protein represents a useful compound binding target and has utility in the identification of new GRM2 compounds having a preferred specificity profile and greater efficacy.

In some embodiments, GRM2sv1 activity is modulated by a ligand compound to achieve one or more of the following: prevent or reduce the risk of occurrence, or recurrence where the potential exist, of seizures, Alzheimer's disease, hyperalgesia, and allodynia.

Compounds modulating GRM2sv1 activity include agonists, antagonists, and allosteric modulators. Generally, but not always, GRM2sv1-antagonists and allosteric modulators negatively affecting GRM2sv1 activity will be used to inhibit cAMP decreases caused by GRM2 thereby increase cAMP formation.

GRM2sv1 activity can also be affected by modulating the cellular abundance of transcripts encoding GRM2sv1. Compounds modulating the abundance of transcripts encoding GRM2sv1 include a cloned polynucleotide encoding GRM2sv1 that can express GRM2sv1 in vivo, antisense nucleic acids targeted to GRM2sv1 transcripts and enzymatic nucleic acids, such as ribozymes and RNAi, targeted to GRM2sv1 transcripts.

In some embodiments, GRM2sv1 activity is modulated to achieve a therapeutic effect upon diseases in which neurological characteristics are in need of adjustment in a subject. For example, epilepsy can be treated by modulating GRM2sv1 activity to achieve, for instance, decreased frequency of convulsions. In other embodiments, the risk of developing Alzheimer's disease is reduced by modulating GRM2sv1 activity to achieve, for example, protection of neurons against β-amyloid peptide-induced apoptosis.

### GRM2sv1 Nucleic Acid

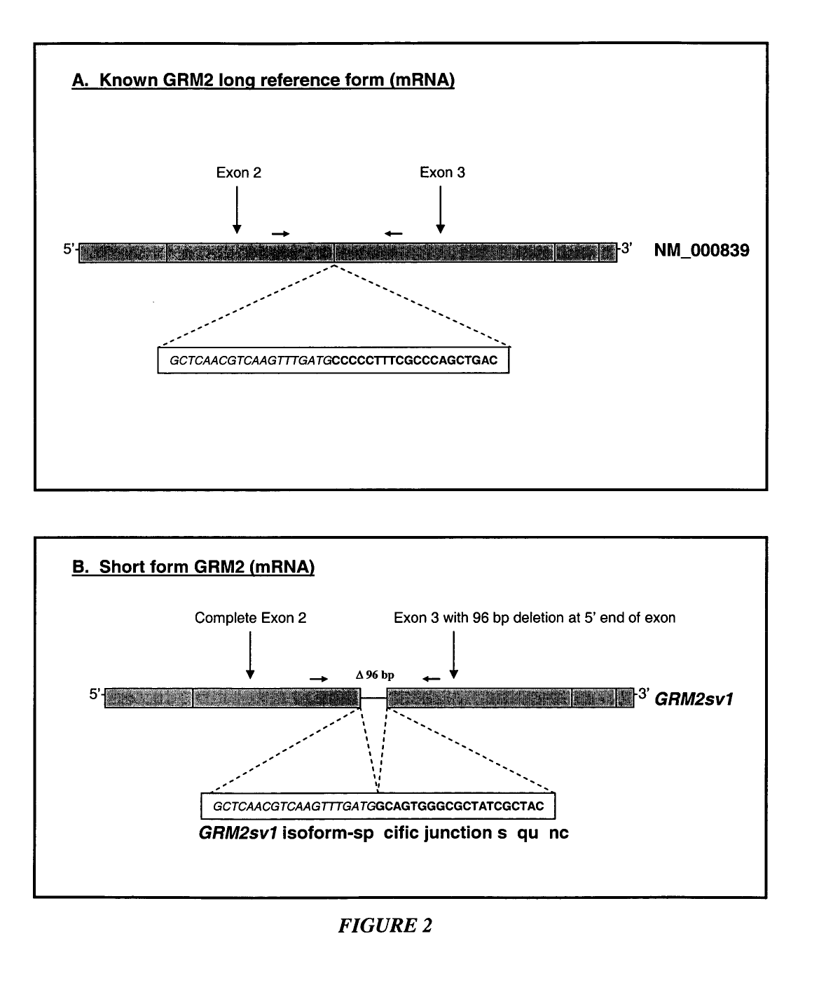

GRM2sv1 nucleic acid contains a region that encodes for a polypeptide comprising, consisting or consisting essentially of SEQ ID NO 2 or comprises, consists, or consists essentially of SEQ ID NO 1. GRM2sv1 nucleic acid has a variety of uses, such as being used as a hybridization probe or a PCR primer to identify the presence of GRM2sv1 nucleic acid; being used as a hybridization probe or PCR primer to identify nucleic acid encoding for proteins related to GRM2sv1; and/or being used for recombinant expression of GRM2sv1 polypeptides. In particular, GRM2sv1 polynucleotides do not have the first 96 nucleotides of exon 3 of GRM2 (see FIG. 2A). This corresponds to a deletion of 32 amino acids from the GRM2 polypeptide.

Regions in GRM2sv1 nucleic acid that do not encode for GRM2sv1 amino acids or are not found in SEQ ID NO 1, if present, are preferably chosen to achieve a particular purpose. Examples of additional regions that can be used to achieve a particular purpose include capture regions that can be used as part of a sandwich assay, reporter regions that can be probed to indicate the presence of the nucleic acid, expression vector regions, and regions encoding for other polypeptides.

The guidance provided in the present application can be used to obtain the nucleic acid sequence encoding for GRM2sv1 related proteins from different sources. Obtaining nucleic acids encoding for GRM2sv1 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

GRM2sv1 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

GRM2sv1 Probes

Probes for GRM2sv1 contain a region that can specifically hybridize to GRM2sv1 target nucleic acid under appropriate hybridization conditions and can distinguish GRM2sv1 nucleic acid from non-target nucleic acids, in particular GRM2 polynucleotides encoding the first 96 nucleotides of exon 3. Probes for GRM2sv1 can also contain nucleic acid that are not complementary to GRM2sv1 nucleic acid.

In embodiments where, for example, GRM2sv1 polynucleotide probes are used in hybridization assays to specifically detect the presence of GRM2sv1 polynucleotides in a sample, the GRM2sv1 polynucleotides comprise at least 16 nucleotides of the GRM2sv1 sequence that correspond to a junction polynucleotide region created by the alternative splicing of exon 2 to nucleotide 97 of exon 3 of the primary transcript the GRM2 gene (see FIG. 2B). For example, the polynucleotide sequence: 5′ AAGTTTGATGGCAGTGGGCG 3′ [SEQ ID NO 3] represents one embodiment of such an inventive GRM2sv1 polynucleotide wherein a first 10 nucleotides region is complementary and hybridizable to the 3′ end of exon 2 of the GRM2 gene and a second 10 nucleotide region is complementary and hybridizable to the novel 5′ end of exon 3 that is missing the first 96 nucleotides of exon 3 of the GRM2 gene (see FIG. 2B). In some embodiments, at least 16 nucleotides of GRM2sv1 comprises a first continuous region of 5 to 11 nucleotides that is complementary and hybridizable to the 3′ end of exon 2 and a second continuous region of 5 to 11 nucleotides that is complementary and hybridizable to the 5′ end of GRM2 exon 3 lacking the first 96 nucleotides.

In other embodiments, the GRM2sv1 polynucleotide comprise at least 40, 60, 80 or 100 nucleotides of the GRM2sv1 sequence that correspond to a junction polynucleotide region created by the alternative splicing of exon 2 to nucleotide position 97 of exon 3 of the primary transcript of the GRM2 gene. In each case the GRM2sv1 polynucleotide is selected to comprise a first continuous region of at least 5 to 11 nucleotides that is complementary and hybridizable to the 3′ end of GRM2 exon 2 and a second continuous region of at least 5 to 11 nucleotides that is complementary and hybridizable to the 5′ end of GRM2 exon 3 lacking the first 96 nucleotides. As will be apparent to a person of skill in the art, a large number of different polynucleotide sequences from the region of the GRM2sv1 exon 2 to exon 3 splice junction may be selected which will, under appropriate hybridization conditions, have the capacity to detectably hybridize to GRM2sv1 polynucleotides and yet will hybridize to a much less extent or not at all to GRM2 isoform polynucleotides wherein GRM2 exon 2 is not spliced to nucleotide 97 of GRM2 exon 3.

Preferably, non-complementary nucleic acid that is present has a particular purpose such as being a reporter sequence or being a capture sequence. However, additional nucleic acid need not have a particular purpose as long as the additional nucleic acid does not prevent the GRM2sv1 nucleic acid from distinguishing between target polynucleotides, e.g., GRM2sv1 polynucleotides and non-target polynucleotides, including, but not limited to GRM2 polynucleotides not comprising a region representing the splice junction of GRM2 exon 2 to nucleotide 97 of GRM2 exon 3 found in GRM2sv1.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

GRM2sv1 polynucleotides, such as those comprising SEQ ID NO 1, can be used to make GRM2sv1 polypeptides. In particular, GRM2sv1 polypeptides can be expressed from recombinant nucleic acid in a suitable host or in vitro using a translation system. Recombinantly expressed GRM2sv1 polypeptides can be used, for example, in assays to screen for compounds that bind to or interact with GRM2sv1. Alternatively, GRM2sv1 polypeptides can also be used to screen for compounds that bind to or interact with one or more GRM2 isoforms but do not bind to or interact with GRM2sv1.

In some embodiments, expression is achieved in a host cell using an expression vector. An expression vector contains recombinant nucleic acid encoding for a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-1(8-2) (ATCC 37110), pdBPV-MMTneo (342-12) (ATCC 37224), pRSVgpt (ATCC 37199), pRSVneo (ATCC 37198), pSV2-dhfr (ATCC 37146) and pUCTag (ATCC 37460), and. Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen) and pYES2 (Invitrogen), Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) and MRC-5 (ATCC CCL 171).

To enhance expression in a particular host it may be useful to modify the sequence provided in SEQ ID NO 1 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acid encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection.

### GRM2sv1 Polypeptides

GRM2sv1 polypeptides contain an amino acid sequence comprising, consisting, or consisting essentially of SEQ ID NO 2. GRM2sv1 polypeptides have a variety of uses, such as providing a marker for the presence of GRM2sv1; being used as an immunogen to produce antibodies binding to GRM2sv1; being used as a target to identify compounds binding selectively to the GRM2sv1; or being used in an assay to identify compounds that bind to or interact with one or more isoforms of GRM2 but do not bind to or interact with GRM2sv1.

In chimeric polypeptides containing one or more regions from GRM2sv1 and one or more regions not from GRM2sv1, the region(s) not from GRM2sv1 can be used, for example, to achieve a particular purpose or to produce a polypeptide that can substitute for GRM2sv1 or a fragment thereof. Particular purposes that can be achieved using chimeric GRM2sv1 polypeptides include providing a marker for GRM2sv1 activity, enhancing an immune response, and to decrease cAMP levels thereby changing gene expression.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional GRM2sv1

Functional GRM2sv1 is an isoform of human metabotropic glutamate receptor 2. The identification of the amino acid and nucleic acid sequences of GRM2sv1 provide tools for obtaining functional proteins related to GRM2sv1 from other sources, for producing GRM2sv1 chimeric proteins, and for producing functional derivatives of SEQ ID NO 2.

GRM2sv1 polypeptides can be readily identified and obtained based on their sequence similarity to GRM2sv1. In particular, GRM2sv1 polypeptides lack 32 amino acids that are encoded by the first 96 nucleotide of exon 3 of the GRM2 gene. Both the amino acid and nucleic acid sequences of GRM2sv1 can be used to help identify and obtain GRM2sv1 polypeptides. For example, SEQ ID NO 2 can be used to produce degenerative nucleic acid probes or primers for identifying and cloning nucleic acid encoding for a GRM2sv1 polypeptide, and SEQ ID NO 1 or fragments thereof, can be used under conditions of moderate stringency to identify and clone nucleic acid encoding GRM2sv1 polypeptides from a variety of different organisms.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with GRM2sv1 obtained from a particular source, derivatives can be produced. Such derivatives include polypeptides with amino acid substitutions, additions and deletions. Changes to GRM2sv1 to produce a derivative having essentially the same properties should be made in a manner not altering the tertiary structure of GRM2sv1.

Differences in naturally occurring amino acids are due to different R groups. An R group effects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

GRM2sv1 Antibodies

Antibodies recognizing GRM2sv1 can be produced using a polypeptide containing SEQ ID NO 2 or a fragment thereof as an immunogen. Preferably, a polypeptide used as an immunogen consists of a polypeptide of SEQ ID NO 2 or a SEQ ID NO 2 fragment of at least 10 amino acids in length encoded by the polynucleotide region representing the junction resulting from the splicing of exon 2 to amino acid 33 of exon 3 of the GRM2 gene.

In some embodiments where, for example, GRM2sv1 polypeptides are used to develop antibodies that bind specifically to GRM2sv1 and not to other isoforms of GRM2, the GRM2sv1 polypeptides comprise at least 8 amino acids of the GRM2sv1 polypeptide sequence encoded by a junction polynucleotide region created by the alternative splicing of exon 2 to nucleotide 97 of exon 3 of the primary transcript the GRM2 gene (see FIG. 2B). For example, the amino acid sequence: amino terminus-NVKFDGSGRY-carboxy terminus [SEQ ID NO 4], represents one embodiment of such an inventive GRM2sv1 polypeptide wherein a first 5 amino acid region is encoded by a nucleotide sequence at the 3′ end of exon 2 of the GRM2 gene and a second 5 amino acid region is encoded by a polynucleotide region beginning at position 97 of exon 3 of the GRM2 gene (see FIG. 2B). Preferably, at least 10 amino acids of the GRM2sv1 polypeptide comprises a first continuous region of 2 to 8 amino acids that are encoded by nucleotides at the 3′ end of exon 2 and a second continuous region of 2 to 8 amino acids that are encoded by nucleotides at the 5′ end of exon 3 beginning at nucleotide position 97 of GRM2 exon 3.

In other embodiments, GRM2sv1-specific antibodies are made using a GRM2sv1 polypeptide that comprises at least 20, 30, 40 or 50 amino acids of the GRM2sv1 sequence that correspond to a junction polynucleotide region created by the alternative splicing of exon 2 to nucleotide position 97 of exon 3 of the primary transcript the GRM2 gene. In each case the GRM2sv1 polypeptide is selected to comprise a first continuous region of at least 5 to 15 amino acids that are encoded by nucleotides at the 3′ end of exon 2 and a second continuous region of 5 to 15 amino acids that are encoded by nucleotides beginning at position 97 of exon 3 of the primary transcript the GRM2 gene.

Antibodies to GRM2sv1 have different uses such as being used to identify the presence of GRM2sv1 and to isolate GRM2sv1 polypeptides. Identifying the presence of GRM2sv1 can be used, for example, to identify cells producing GRM2sv1. Such identification provides an additional source of GRM2sv1 and can be used to distinguish cells known to produce GRM2sv1 from cells that do not produce GRM2sv1. For example, antibodies to GRM2sv1 can distinguish human cells expressing GRM2sv1 from human cells not expressing GRM2sv1 or non-human cells (including bacteria) that do not express GRM2sv1. Such GRM2sv1 antibodies can also be used to determine the effectiveness of GRM2sv1 ligands, using techniques well known in the art, to detect and quantify changes in the protein levels of GRM2sv1 in cellular extracts, and in situ immunostaining of cells and tissues.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

GRM2sv1 Binding Assay

GRM2sv1 or a fragment thereof can be used in binding studies to identify compounds binding to the protein. In another embodiment, the GRM2sv1 or a fragment thereof can be used in binding studies with GRM2 or a fragment thereof, to identify compounds that: bind to both GRM2sv1 and GRM2; bind to GRM2sv1 and not GRM2 or a GRM2 isoform that is not GRM2sv1; or bind to or interact with one or more GRM2 isoforms and not with GRM2sv1. Such studies can be performed using different formats including competitive and non-competitive formats. Further competition studies can be carried out using additional compounds determined to bind to GRM2sv1 or GRM2.

The particular GRM2sv1 sequence involved in ligand binding can be readily identified by using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

Preferably, binding studies are performed using GRM2sv1 expressed from a recombinant nucleic acid. More preferably, recombinantly expressed GRM2sv1 consists of the SEQ ID NO 2 amino acid sequence.

Binding assays can be performed using individual compounds or preparations containing different numbers of compounds. A preparation containing different numbers of compounds having the ability to bind to GRM2sv1 can be divided into smaller groups of compounds that can be tested to identify the compound(s) binding to GRM2sv1.

Binding assays can be performed using recombinantly produced GRM2sv1 present in different environments. Such environments include, for example, cell extracts and purified cell extracts containing a GRM2sv1 recombinant nucleic acid; and also include, for example, the use of a purified GRM2sv1 polypeptide produced by recombinant means which is introduced into a different environment.

In one embodiment of the invention, a binding method is provided for screening for a ligand able to bind selectively to GRM2sv1 polypeptides. The method comprises the steps: providing a GRM2sv1 polypeptide comprising SEQ ID NO 2; providing a GRM2 isoform polypeptide that is not GRM2sv1, contacting the GRM2sv1 polypeptide and the GRM2 isoform polypeptide that is not GRM2sv1 with a test preparation comprising one or more test ligand; and then determining the binding of the test preparation to the GRM2sv1 polypeptide and the GRM2 isoform polypeptide that is not GRM2sv1, wherein a ligand which binds the GRM2sv1 polypeptide but does not bind the GRM2 isoform polypeptide that is not GRM2sv1 is a ligand that selectively binds the GRM2sv1 polypeptide.

In another embodiment of the invention, a binding method is provided for screening for a ligand able to bind selectively to a GRM2 isoform polypeptide that is not GRM2sv1. The method comprises the steps: providing a GRM2sv1 polypeptide comprising SEQ ID NO 2; providing a GRM2 isoform polypeptide that is not GRM2sv1, contacting the GRM2sv1 polypeptide and the GRM2 isoform polypeptide that is not GRM2sv1 with a test preparation comprising one or more test ligands; and then determining the binding of the test preparation to the GRM2sv1 polypeptide and the GRM2 isoform polypeptide that is not GRM2sv1, wherein a ligand which binds the GRM2 isoform polypeptide that is not GRM2sv1 but does not bind the GRM2sv1 polypeptide is a ligand that selectively binds the GRM2 isoform polypeptide that is not GRM2sv1.

The above-described selective binding assays can also be performed with a polypeptide fragment of GRM2sv1, wherein the polypeptide fragment comprises at least 10 consecutive amino acids that are encoded by a nucleotide sequence that bridge the junction created by the splicing of the 3′ end of exon 2 to the nucleotide position 97 of exon 3 of a transcript of the GRM2 gene. Similarly, the selective binding assays may also be performed using a polypeptide fragment of a GRM2 isoform polypeptide that is not GRM2sv1 wherein the polypeptide fragment comprises at least 10 consecutive amino acids that are encoded by: a) a nucleotide sequence that is contained within the first 96 nucleotides of exon 3 of GRM2; or b) a nucleotide sequence that bridges the junction created by the splicing of the 3′ end of exon 2 to the 5′ end of exon 3 of a transcript of the GRM2 gene.

GRM2 Functional Assays

The identification of GRM2sv1 as a splice variant of GRM2 provides a means for screening for ligands that bind to GRM2sv1 protein thereby altering the ability of the GRM2sv1 polypeptide to bind to glutamate and/or any other reaction intermediate compound, to bind to any agonist, or to perform as a metabotropic glutamate receptor. Assays involving a functional GRM2sv1 polypeptide can be employed for different purposes such as selecting for ligands active at GRM2sv1, evaluating the ability of a ligand to effect glutamate binding activity, and mapping the activity of different GRM2sv1 regions. GRM2sv1 activity can be measured using different techniques such as: detecting a change in the intracellular conformation of GRM2sv1; detecting a change in the intracellular location of GRM2sv1; detecting the amount of binding of glutamate or other agonist to GRM2sv1; measuring the changes in level of cAMP activity caused by GRM2sv1; or measuring phosphoinositide hydrolysis.

Recombinantly expressed GRM2sv1 can be used to facilitate determining whether a ligand is active at GRM2sv1. For example, GRM2sv1 can be expressed by an expression vector in a cell line and used in a co-culture growth assay, such as described in WO 99/59037, to identify ligands that bind to GRM2sv1.

A large variety of assays can be used to investigate the properties of GRM2 and therefore would also be applicable to the measurement of GRM2sv1 function. These include techniques for measuring cAMP levels, glutamate binding activity, and Ca²⁺ levels (Litschig et al., 1999 Molecular Pharmacology, 55:453-461). Phosphoinositide hydrolysis assays can also be used to determine the functionality of a GRM2 isoform protein or GRM2sv1 by measuring the accumulation of tritiated inositol monophosphate ([³H]-IP₁) in the presence of LiCl (Berridge, 1983 Biochem. J., 212:849-858).

GRM2sv1 functional assays can be performed using cells expressing GRM2sv1 at a high level contacted with individual test ligands or preparations containing two or more different ligands. A preparation containing different ligands where one or more ligands affect GRM2sv1 in cells over producing GRM2sv1 as compared to control cells containing expression vector lacking GRM2sv1 coding sequence, can be divided into smaller groups of ligands to identify the compound(s) affecting GRM2sv1 activity.

GRM2sv1 functional assays can be performed using recombinantly produced GRM2sv1 present in different environments. Such environments include, for example, cell extracts and purified cell extracts containing the GRM2sv1 expressed from recombinant nucleic acid and an appropriate membrane for the polypeptide; and the use of a purified GRM2sv1 produced by recombinant means that is introduced into a different environment suitable for measuring glutamate binding.

### Modulating GRM2sv1 Expression

GRM2sv1 expression can be modulated as a means for increasing or decreasing GRM2sv1 activity. Such modulation includes inhibiting GRM2sv1 nucleic acid activity to reduce GRM2sv1 expression or supplying GRM2sv1 nucleic acid to increase GRM2sv1 activity.

Inhibition of GRM2sv1 Activity

GRM2sv1 nucleic acid activity can be inhibited using nucleic acids recognizing GRM2sv1 nucleic acid and affecting the ability of such nucleic acid to be transcribed or translated. Inhibition of GRM2sv1 nucleic acid activity can be used, for example, in target validation studies.

A preferred target for inhibiting GRM2sv1 is mRNA translation. The ability of mRNA encoding GRM2sv1 to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNAi also can be used to prevent protein expression of a target transcript. This method is based on the interfering properties of double-stranded RNA derived from the coding regions of gene that disrupts the synthesis of protein from transcribed RNA.

Enzymatic nucleic acid can recognize and cleave another nucleic acid molecule. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can affect certain anti-sense activities such as the ability to be cleaved by RNAse H, and can affect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999 Cell 99:123-32; Fire, et al., 1998 Nature 391:806-11), plants (Hamilton and Baulcombe, 1999 Science 286:950-52), Drosophila (Hammond, et al., 2001 Science 293:1146-50; Misquitta and Patterson, 1999 Proc. Nat. Acad. Sci. 96:1451-56; Kennerdell and Carthew, 1998 Cell 95:1017-26), and mammalian cells (Bernstein, et al., 2001 Nature 409:363-6; Elbashir, et al., 2001 Nature 411:494-8).

Increasing GRM2sv1 Expression

Nucleic acid coding for GRM2sv1 can be used, for example, to cause a change in cAMP levels or to create a test system (e.g., a transgenic animal) for screening for ligands affecting GRM2sv1 expression. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of GRM2sv1

To identify variants of the “normal” splicing of the exon regions encoding GRM2, a series of RT-PCR reactions were designed to represent all of the exon junctions of the GRM2 gene. PolyA purified mRNA isolated from 79 different human tissues was obtained from BD Biosciences Clontech (Palo Alto, Calif.), Biochain Institute, Inc. (Hayward, Calif.), and Ambion Inc. (Austin, Tex.). In addition, one monkey mRNA sample (brain, from Biochain Institute, Inc.) was also obtained and assayed. Primers were designed using a Primer3 program (Whitehead Institute for Biomedical Research, Cambridge, Mass.) to span the junction between: a) exon 1 and exon 2, b) exon 2 and exon 3, and c) exon 3 and exon 5. The GRM2₁₋₂ primer set (exon 1 forward primer: 5′ AGAGGACTGTGGTCCTGTCAATGAG 3′ [SEQ ID NO 5] and exon 2 reverse primer: 5′ ACACATAGGTCCAGTTGAAGAAGCG 3′ [SEQ ID NO 6]) was expected to amplify a PCR product of 482 basepairs. The GRM2₂₋₃ primer set (exon 2 forward primer: 5′ TCCAAGATCATGTTTGTGGTCAATG 3′ [SEQ ID NO 7] and exon 3 reverse primer: 5′ AGTGAATTCGTCCAATCGGTACTCA 3′ [SEQ ID NO 8]) was expected to amplify a PCR product of 483 basepairs. The GRM2₃₋₅ primer set (exon 3 forward primer: 5′CTGCACGCTT TATGCCTTCAATACT 3′ [SEQ ID NO 9] and exon 5 reverse primer: 5′CATTGCAAACAG TGGGGACAAACT 3′ [SEQ ID NO 10]) was expected to amplify a PCR product of 359 basepairs.

Twenty-five nanograms (ng) of polyA mRNA from each tissue was subjected to a one-step reverse transcription-PCR amplification protocol using the Qiagen, Inc. (Valencia, Calif.), One-Step RT-PCR kit, using the following conditions:

Cycling conditions were as follows:

```
50° C. for 30 minutes;
95° C. for 15 minutes;
35 cycles of:
  94° C. for 30 seconds;
  62.5° C. for 40 seconds;
  72° C. for 1 minutes; then
72° C. for 10 minutes.
```

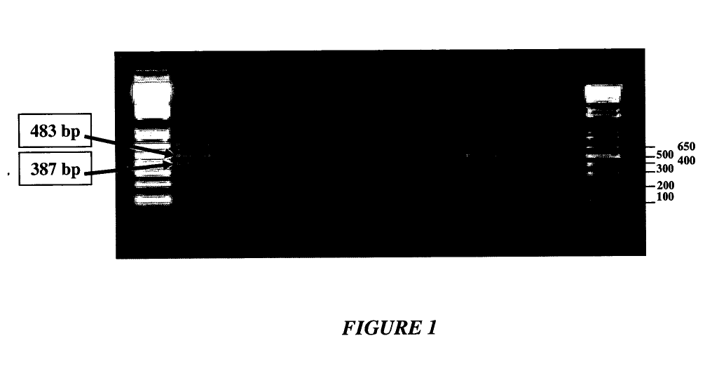

RT-PCR amplification products (amplicons) were size fractionated on a 2% agarose gel. FIG. 1 shows the separation of RT-PCR products amplified from the ten tissues listed in Table 1. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

TABLE 1

| Lane | |
|---|---|
| number | Sample |
| 1 | Testes |
| 2 | Epididymus |
| 3 | Uterus |
| 4 | Uterus, corpus |
| 5 | Placenta |
| 6 | Ovary |
| 7 | Spleen |
| 8 | Thymus |
| 9 | Lymph node |
| 10 | Peripheral leukocytes |

Four different RT-PCR amplicons were obtained from human testes mRNA samples using the GRM2₂₋₃ primer set (FIG. 1, lane 1), while two different RT-PCR amplicons were obtained from human thymus mRNA samples using the GRM2₂₋₃ primer set (FIG. 1, lane 8). Twenty-six human tissues, predominantly in the central nervous system, exhibited the expected amplicon size of 483 basepairs for the normally spliced reference GRM2 mRNA (FIG. 1 and Table 2). Table 2 presents the complete list of the 79 human polyA mRNA samples and one monkey mRNA sample that were used in the reverse transcription, polymerase chain reaction (RT-PCR) reactions to confirm the presence of a novel form of GRM2. Table 2 also lists whether the long reference form GRM2 transcript or the short form GRM2sv1 was detected in each tissue. A plus (+) symbol indicates that the transcript was detected, while a minus (−) symbol indicates that the transcript was not detected. Out of the 80 samples analyzed, the GRM2 short variant form, GRM2sv1, was present in only two samples: testes and thymus. However, the GRM2 long reference form polynucleotide was detected in 26 tissues.

TABLE 2

| SAMPLE | GRM2 | GRM2sv1 |
|---|---|---|
| HEART | − | − |
| HEART, AORTA | − | − |
| HEART, ATRIOVENTRIVCULAR | − | − |
| NODES | | |
| HEART, INTER VENTRICULAR | − | − |
| SEPTUM | | |
| FETAL HEART - BIOCHAIN | − | − |
| TONGUE | − | − |
| TONSIL | − | − |
| SALIVARY GLAND | − | − |
| TRACHEA | − | − |
| STOMACH | − | − |
| SMALL INTESTINE | − | − |
| PANCREAS | − | − |
| DUODENUM | − | − |
| JEJUNUM | − | − |
| ILEUM | − | − |
| ILEOCECUM | − | − |
| TRANSVERSE COLON | − | − |
| DESCENDING COLON | − | − |
| RECTUM | − | − |
| KIDNEY | − | − |
| KIDNEY, FETAL | − | − |
| LIVER | − | − |
| LIVER, FETAL | − | − |
| LIVER, LEFT LOBE | − | − |
| HUMAN BLADDER | − | − |
| ADRENAL GLAND | − | − |
| ADRENAL CORTEX | − | − |
| ADRENAL MEDULLA | − | − |
| THYROID | − | − |
| PROSTATE | − | − |
| TESTES | + | + |
| EPIDIDYMUS | − | − |
| UTERUS | − | − |
| UTERUS, CORPUS | − | − |
| PLACENTA | − | − |
| OVARY - AMBION | − | − |
| SPLEEN | + | − |
| THYMUS | + | + |
| LYMPH NODE | − | − |
| PERIPHERAL LEUKOCYTES | + | − |
| BONE MARROW | − | − |
| LUNG | − | − |
| LUNG, FETAL | − | − |
| LUNG, UPPER RIGHT LOBE | − | − |
| ADIPOSE TISSUE | − | − |
| RETINA | + | − |
| SKELETAL MUSCLE | − | − |
| SKELETAL MUSCLE, FETAL - | − | − |
| BIOCHAIN | | |
| VERTEBRA, FETAL | − | − |
| HELA CELL (S3) | − | − |
| LEUKEMIA PROMYELOCYTIC | − | − |
| (HL-60) | | |
| BURKITT'S LYMPHOMA (DAUDI) | − | − |
| LEUKEMIA CHRONIC | − | − |
| MYELOGENOUS (K562) | − | − |
| COLORECTAL | − | − |
| ADENOCARCINOMA | − | − |
| BURKITT'S LYMPHOMA (RAJI) | − | − |
| MELANOMA (G361) | − | − |
| LUNG CARCINOMA (A549) | − | − |
| BRAIN | + | − |
| BRAIN, FETAL | + | − |
| BRAIN, AMYGDALA | + | − |
| BRAIN, CAUDATE NUCLEUS | − | − |
| BRAIN, CORPUS CALLOSUM | − | − |
| BRAIN, THALAMUS | + | − |
| BRAIN, CEREBELLUM | + | − |
| BRAIN, CEREBRAL CORTEX | + | − |
| BRAIN, HIPPOCAMPUS | + | − |
| BRAIN, POSTCENTRAL GYRUS | + | − |
| BRAIN, FRONTAL LOBE | + | − |
| BRAIN, MEDULLA OBLONGATA | + | − |
| BRAIN, OCCIPITAL LOBE | + | − |
| BRAIN, PARIENTAL LOBE | + | − |
| BRAIN, PONS | + | − |
| BRAIN, PUTAMEN | + | − |
| BRAIN, TEMPORAL LOBE | + | − |
| BRAIN, HYPOTHALAMUS | + | − |
| BRAIN, NUCLEUS ACCUMBENS | + | − |
| BRAIN, PARACENTRAL GYRUS | + | − |
| SPINAL CHORD | + | − |
| SPINAL CHORD, FETAL | + | − |
| MONKEY BRAIN | + | − |

In addition, the monkey brain mRNA sample (Table 2 and data not shown) also exhibited the expected 483 basepair amplicon. However, in addition to the expected GRM2 amplicon of 483 basepairs, human testes and thymus tissues also exhibited a second amplicon of 387 basepairs (FIG. 1). Interestingly, testes mRNA samples (FIG. 1, lane 1), appeared to exhibit four different GRM2 mRNA forms; the long reference form, the short form, and two larger amplicons of approximately 550 basepairs and 600 basepairs. The two larger amplicons were only observed in mRNA derived from human testes tissue.

Sequence analysis of the 387 basepair amplicon of GRM2 revealed that this amplicon form is due to splicing of exon 2 of the GRM2 hnRNA nucleotide 97 of exon 3. That is, the short form GRM2 amplicon is due to the deletion of 96 nucleotides at the 5′ end of the exon 3 coding sequence of GRM2.

### Example 2

### Cloning of GRM2sv1

RT-PCR data indicate that in addition to the normal reference GRM2 mRNA sequence, NM_{—}000839, encoding GRM2 protein, NP_{—}000830), a splice variant form of GRM2 mRNA also exists in testes and thymus tissues. Indeed, inspection of the amplicon band intensities in FIG. 1, suggests that the GRM2 short form of the GRM2 mRNA is present in an amount that is about equal to or slightly less than the “reference” exon 3 containing GRM2 mRNA in testes, and approximately one-third the abundance of the normal exon 3 mRNA in thymus.

A full length GRM2 clone having a nucleotide sequence comprising the “short” form splice variant (hereafter referred to as GRM2sv1) as identified in Example 2 is isolated using a 5′ “forward” GRM2 primer and a 3′ “reverse” GRM2 primer, to amplify and clone the entire GRM2sv1 mRNA coding sequence. The 5′ “forward” GRM2 primer is designed to have a nucleotide of 5′ ATGGGATCGCTGCTTGCGCTCCTGG 3′ [SEQ ID NO 11]. The 3′ “reverse” GRM2 primer is designed to have the nucleotide sequence of 5′ AAGCGATGACG TTGTCGAGTCCTCACG 3′ [SEQ ID NO 12].

RT-PCR

The GRM2sv1 cDNA sequence is cloned using a combination of reverse transcription (RT) and polymerase chain reaction (PCR). More specifically, about 25 ng of testes polyA mRNA (Ambion, Austin, Tex.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clonetech (PaloAlto, Calif.) Advantage 2 PCR kit. PCR is done in a Gene Amp PCR System 9700 (Applied Biosystems, Foster City, Calif.) using the GRM2 “forward” and “reverse” primers. After an initial 94° C. denaturation of 1 minute, 35 cycles of amplification are performed using a 30 second denaturation at 94° C. followed by a 1 minute annealing at 65° C. and a 90 second synthesis at 68° C. The 35 cycles of PCR are followed by a 7 minute extension at 68° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determined if the PCR has yielded products of the expected size, in the case of the predicted GRM2sv1 mRNA, a product of about 2.5 kilobases (Kb). The remainder of the 50 pi PCR reactions from testes is purified using the QIAquik Gel extraction Kit (Qiagen, Valencia, Calif.) following the QIAquik PCR Purification Protocol provided with the kit. An about 50 μl of product obtained from the purification protocol is concentrated to about 6 μl by drying in a Speed Vac Plus (SC 1 A, from Savant, Holbrook, N.Y.) attached to a Universal Vacuum Sytem 400 (also from Savant) for about 30 minutes on medium heat.

Cloning of RT-PCR Products

About 4 μl of the 6 μl of purified GRM2sv1 RT-PCR product from testes is used in a cloning reaction using the reagents and instructions provided with the TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative GRM2sv1 clones are identified and prepared for a PCR reaction to confirm the presence of the polynucleotide amplicon expected to result from the splicing of the 3′ end of GRM2 exon 2 to nucleotide position 97 of exon 3 of GRM2. A 25 μl PCR reaction is performed as described above (RT-PCR section) to detect the presence of GRM2sv1, except that the reaction includes miniprep DNA from the TOPO TA/GRM2 ligation as a template, and uses the GRM2₂₋₃ primer set. About 10 μl of each 25 μl PCR reaction is run on a 1% Agarose gel and the DNA bands generated by the PCR reaction are visualized and photographed on a UV light box to determine which minipreps samples have a PCR product of the size predicted for the predicted corresponding GRM2sv1 splice variant mRNA. Clones having the GRM2sv1 structure are identified based upon amplification of an amplicon band of 387 basepairs, whereas a normal reference GRM2 clone will give rise to an amplicon band of 483 basepairs. DNA sequence analysis of the GRM2sv1 cloned DNA produces a polynucleotide sequence having a GRM2sv1 coding sequence of SEQ ID NO 1.

SEQ ID NO 1 contains an open reading frame that encodes a GRM2sv1 protein (SEQ ID NO 2) identical to the reference protein GRM2 (NP_{—}000830), but lacking a 32 amino acid region encoded by the 5′ end portion of exon 3 of the full length coding sequence of the reference GRM2 mRNA (NM_{—}000839).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified human nucleic acid comprising SEQ ID NO 1, or the complement thereof.

2. The purified nucleic acid of claim 1, wherein said nucleic acid comprises a sequence encoding SEQ ID NO 2.

3. The purified nucleic acid of claim 1, wherein said nucleic acid encodes a polypeptide consisting of SEQ ID NO 2.

4. A purified polypeptide comprising SEQ ID NO 2.

5. The polypeptide of claim 4, wherein said polypeptide consists of SEQ ID NO 2.

6. An expression vector comprising a nucleotide sequence encoding SEQ ID NO 2, wherein said nucleotide sequence is transcriptionally coupled to an exogenous promoter.

7. The expression vector of claim 6, wherein said nucleotide sequence encodes a polypeptide consisting of SEQ ID NO 2.

8. The expression vector of claim 6, wherein said nucleotide sequence comprises SEQ ID NO 1.

9. The expression vector of claim 6, wherein said nucleotide sequence consists of SEQ ID NO 1.

10. A method of screening for a compound that is able to bind selectively to GRM2sv1 comprising the steps of:
(a) providing a GRM2sv1 polypeptide comprising SEQ ID NO 2;
(b) providing one or more GRM2 isoform polypeptides that are not GRM2sv1,
(c) contacting said GRM2sv1 polypeptide and said GRM2 polypeptide that is not GRM2sv1 with a test preparation comprising one or more test compounds; and
(d) determining the binding of said test preparation to said GRM2sv1 polypeptide and said GRM2 polypeptide that is not GRM2sv1, wherein a compound that binds said GRM2sv1 polypeptide but does not bind said GRM2 polypeptide that is not GRM2sv1 is a compound that selectively binds said GRM2sv1 polypeptide.

11. The method of claim 10, wherein said GRM2sv1 polypeptide is obtained by expression of said polypeptide from an expression vector comprising a polynucleotide encoding SEQ ID NO 2.

12. The method of claim 11, wherein said polypeptide consists of SEQ ID NO 2.

13. The method of claim 10, wherein said steps (b) and (c) are performed in vitro.

14. The method of claim 10, wherein said steps (a), (b) and (c) are preformed using a whole cell.

15. The method of claim 10, wherein said test preparation contains one compound.
